(19) United States
(12) Patent Application Publication
(10) Pub. No.: US 2005/0282836 A1
(43) Pub. Date: Dec. 22, 2005

(54) Solid orally ingestible formulations of tetrodotoxin

(76) Inventor: Weiyang Lin, Vancouver (CA)

Correspondence Address: BIRCH STEWART KOLASCH & BIRCH, PO BOX 747, FALLS CHURCH, VA, 22040-0747, US

(21) Appl. No.: 10/872,528

(22) Filed: Jun. 22, 2004

(51) Int. Cl. A61K 31/519

(52) U.S. Cl. 514/267

(57) ABSTRACT

The present invention refers to outwardly solid or completely solid oral (or designed to be orally ingested) formulations of tetrodotoxin and/or analogs or derivatives thereof.

### FIELD OF THE INVENTION

The present invention refers to solid oral (or designed to be orally ingested) formulations of Tetrodotoxin.

### BACKGROUND OF THE INVENTION

Tetrodotoxin (alternatively in the context of this invention abbreviated TTX), also known as Ti Qu Duo Xin, is an alkaloid found in puffer fish (Tetradontiae). The chemical name is Octahydro-12-(Hydroxymethyl)-2-imino-5,9,7,10a-dimethano-10aH-[1,3]dioxocino[6,5-d]pyrimidine-4,7,10,11,12-pentol with a Molecular formula C11H17N3O8 and a Molecular weight of: 319,27. It is a potent non-protein neurotoxin and an indispensable tool drug for the study of neurobiology and physiology. Tetrodotoxin (TTX) is a marine organic toxin which is mainly found in testicles, ovaries, eggs, livers, spleens, eyeballs, and blood of puffer fish as well as in diverse animal species, including goby fish, newt, frogs and the blue ringed octopus and even in marine alga. It is a well established substance with production processes known. Usually TTX is extracted from marine organisms (e.g. JP 270719 Got and Takahsi) but besides numerous others methods of synthesis are also described (and used for the preparation of tetrodotoxin in connection to this invention) in U.S. Pat. No. 6,552,191, U.S. Pat. No. 6,478,966, U.S. Pat. No. 6,562,968 or 2002/0086997 all of those are included here by reference. For one of the many descriptions of TTX it is recommended turn to e.g. Tu, Anthony (Ed.) Handbook of Natural Toxins, Vol. 3: Marine Toxins and Venoms, 1988, 185-210 as well as Kao (1966), Pharmacol. Rev. 18:997-1049 and others.

Tetrodotoxin is a well known compound described for example in WO02/22129 as systemically acting as analgesic. Usually as described in the examples as well as in those of CN 1145225 for this method of treatment a parenteral solution is prepared and applied to the patient. Aside from that WO 03/099301 describes formulations administered through the respiratory tract which are in the form of an aerosol, spray or gasoloid formulation and as another parenteral route CN 1227102 describes a bucal tablet to be used in addiction treatment.

Older journals describe that based on the method described by Tahara in U.S. Pat. No. 1,058,643, there was a product sold in Japan containing a 1% solution of TTX extract for uses such as enuresis without stating the application route (Iwakawa and Kimura, Archiv fuer Experimentelle Pathologie und Pharmakologie (1922), 93, 305-31). Even though a majority of the intended uses points at topical application an indication such as enuresis clearly hints at other ways with the oral route given probably self administration being highly likely. In parallel there were trials in the 1930s (Hsiang, Nai Shi; Manshu Igaku Zasshi (1939), 30, 639-47 (German abstr. 179) testing the abilities of TTX for addiction treatment using the oral route but not explaining the formulation used. As this at least coincides with Dr. Tahara's solution it can safely be assumed that the formulation used was a solution as well.

Still, some recent art like WO02/22129 suggests oral application and CN 1145225 even gives a short reference to oral use in a trial devoted to addiction treatment but again the taken “quanannin” is a solution and the description clearly dismisses the oral use stating that “results only became comparable after a dose 50 times that of injection route was applied”. So over the many years and publications any attempts to practically use TTX orally for therapy seems to have been scarce at best obviously being driven by a safety-orientated kind of thinking. A reported 50 times higher dose (CN 1145225) of the known toxin TTX to be necessary has raised concerns in regards to safety (with the therapeutic window being narrowed). Therefore, today there seems to be a clear prejudice in the art, that TTX has to be used parenterally. Especially it was and still is assumed in the art that TTX was not going to be stable if taken orally and definitely not safe enough.

However the present inventor has quite surprisingly found out that TTX is stable in the intestinal fluid and that oral formulations especially solid formulations like a tablet or a capsule are stable and highly effective while being toxicologically safe when ingested.

Therefore the subject of this invention is an outwardly solid formulation designed to be orally ingested containing tetrodotoxin and/or any of its analogs optionally in the form of its racemate, pure stereoisomers, especially enantiomers or diastereomers or in the form of mixtures of stereoisomers, especially enantiomers or diastereomers, in any suitable ratio; in neutral form, in the form of an acid or base or in form of a salt, especially a physiologically acceptable salt, or in form of a solvate, especially a hydrate, especially a hydrate and—(preferably but not necessarily) optionally—at least one auxiliary material and/or additive and/or optionally another active ingredient.

These formulations were stable and highly effective while being toxicologically safe when ingested. The advantage of an oral formulation especially for the systemic treatment of pain is quite obvious with patient compliance rising considerably with an oral formulation compared to injection—at least when not hospitalized. Another obvious advantage is better storage and handling capabilities of solid oral formulations.

In connection with this invention “formulation” means a pharmaceutical formulation or a pharmaceutical composition in which the parts and ingredients including the active compound/s (ingredient/s) or—(preferably but not necessarily) optionally—auxiliary material/s and/or additive/s are designed to be used therapeutically in the treatment of a patient like e.g a tablet, an oral liquid dosage form (e.g. a suspension of particles) or a capsule.

The formulations or pharmaceutical compositions according to the invention contain the active ingredient as well as—(preferably but not necessarily) optionally—at least one auxiliary material and/or additive. The auxiliary material and/or additive can be selected from carrier, excipient, support materials, glidants, fillers, solvents, diluents, colorants, taste conditioners like sugars, antioxidants and/or binders. The selection of these auxiliary materials and/or additives and of the amounts to be used depends upon how the pharmaceutical composition is to be applied. For these oral application preparations in the form of tablets, chewable tablets, dragees, capsules, granules and drops are suitable.

“Capsules” are generally understood in the art as being solid dosage forms in which a drug formulation is enclosed in a soluble container or “shell”. As such, a capsule may be considered as an example of an “outwardly solid form” according to the present invention. Capsules may be hard or soft. Consumable capsules are generally made from gelatin products, but other substances are known.

The shells are usually formed from gelatin; however, they also may be made from starch or other suitable substances. Hard shell capsule sizes range from No. 5, the smallest, to No. 000, which is the largest, except for veterinary sizes. However, size No. 00 generally is the largest size acceptable to patients. Size 0 hard gelatin capsules having an elongated body (known as size OE) also are available, which provide greater fill capacity without an increase in diameter. Hard gelatin capsules consist of two, telescoping cap and body pieces.

Generally, there are unique grooves or indentations molded into the cap and body portions to provide a positive closure when fully engaged, which helps prevent the accidental separation of the filled capsules during shipping and handling. Positive closure also may be affected by spot fusion (“welding”) of the cap and body pieces together through direct thermal means or by application of ultrasonic energy. Factory-filled hard gelatin capsules may be completely sealed by banding, a process in which one or more layers of gelatin are applied over the seam of the cap and body, or by a liquid fusion process wherein the filled capsules are wetted with a hydroalcoholic solution that penetrates into the space where the cap overlaps the body, and then dried. Hard shell capsules made from starch consist of two, fitted cap and body pieces. Since the two pieces do not telescope or interlock positively, they are sealed together at the time of filling to prevent their separation. Starch capsules are sealed by the application of a hydroalcoholic solution to the recessed section of the cap immediately prior to its being placed onto the body.

The banding of hard shell gelatin capsules or the liquid sealing of hard shell starch capsules enhances consumer safety by making the capsules difficult to open without causing visible, obvious damage, and may improve the stability of contents by limiting O2 penetration. Industrially filled hard shell capsules also are often of distinctive color and shape or are otherwise marked to identify them with the manufacturer. Additionally, such capsules may be printed axially or radially with strengths, product codes, etc. Pharmaceutical grade printing inks are usually based on shellac and employ FDA-approved pigments and lake dyes.

In extemporaneous prescription practice, hard shell capsules may be hand-filled; this permits the prescriber a latitude of choice in selecting either a single drug or a combination of drugs at the exact dosage level considered best for the individual patient. This flexibility gives hard shell capsules an advantage over compressed tablets and soft shell capsules as a dosage form. Hard shell capsules are usually formed from gelatins having relatively high gel strength. Either type may be used, but blends of pork skin and bone gelatin are often used to optimize shell clarity and toughness. Hard shell capsules also may be formed from starch or other suitable substances. Hard shell capsules may also contain colorants, such as D&C and FD&C dyes or the various iron oxides, opaquing agents such as titanium dioxide, dispersing agents, hardening agents such as sucrose, and preservatives. They normally contain between 10% and 15% water.

Hard gelatin capsules are made by a process that involves dipping shaped pins into gelatin solutions, after which the gelatin films are dried, trimmed, and removed from the pins, and the body and cap pieces are joined. Starch capsules are made by injection molding a mixture of starch and water, after which the capsules are dried. A separate mold is used for caps and bodies, and the two parts are supplied separately. The empty capsules should be stored in tight containers until they are filled. Since gelatin is of animal origin and starch is of vegetable origin, capsules made with these materials should be protected from potential sources or microbial contamination.

Hard shell capsules typically are filled with powder, beads, or granules. Inert sugar beads (nonpareils) may be coated with active ingredients and coating compositions that provide extended-release profiles or enteric properties. Alternatively, larger dose active ingredients themselves may be suitably formed into pellets and then coated. Semisolids or liquids also may be filled into hard shell capsules; however, when the latter are encapsulated, one of the sealing techniques must be employed to prevent leakage.

In hard gelatin capsule filling operations, the body and cap of the shell are separated prior to dosing. In hard starch shell filling operations, the bodies and caps are supplied separately and are fed into separate hoppers of the filling machine. Machines employing various dosing principles may be employed to fill powders into hard shell capsules; however, most fully automatic machines form powder plugs by compression and eject them into empty capsule bodies. Accessories to these machines generally are available for the other types of fills. Powder formulations often require adding fillers, lubricants, and glidants to the active ingredients to facilitate encapsulation. The formulation, as well as the method of filling, particularly the degree of compaction, may influence the rate of drug release. The addition of wetting agents to the powder mass is common where the active ingredient is hydrophobic. Disintegrants also may be included in powder formulations to facilitate deaggregation and dispersal of capsule plugs in the gut. Powder formulations often may be produced by dry blending; however, bulky formulations may require densification by roll compaction or other suitable granulation techniques.

Powder mixtures that tend to liquefy may be dispensed in hard shell capsules if an absorbent such as magnesium carbonate, colloidal silicon dioxide, or other suitable substance is used. Potent drugs are often mixed with an inert diluent before being filled into capsules. Where two mutually incompatible drugs are prescribed together, it is sometimes possible to place one in a small capsule and then enclose it with the second drug in a larger capsule. Incompatible drugs also can be separated by placing coated pellets or tablets, or soft shell capsules of one drug into the capsule shell before adding the second drug.

Thixotropic semisolids may be formed by gelling liquid drugs or vehicles with colloidal silicas or powdered high molecular weight polyethylene glycols. Various waxy or fatty compounds may be used to prepare semisolid matrices by fusion.

Soft shell capsules made from gelatin (sometimes called softgels) or other suitable material require large-scale production methods. The soft gelatin shell is somewhat thicker than that of hard shell capsules and may be plasticized by the addition of a polyol such as sorbitol or glycerin. The ratio of dry plasticizer to dry gelatin determines the “hardness” of the shell and may be varied to accommodate environmental conditions as well as the nature of the contents. Like hard shells, the shell composition may include approved dyes and pigments, opaquing agents such as titanium dioxide, and preservatives. Flavors may be added and up to 5% sucrose may be included for its sweetness and to produce a chewable shell. Soft gelatin shells normally contain 6% to 13% water. Soft shell capsules also may be printed with a product code, strength, etc. In most cases, soft shell capsules are filled with liquid contents. Typically, active ingredients are dissolved or suspended in a liquid vehicle.

Classically, an oleaginous vehicle such as a vegetable oil was used; however, nonaqueous, water-miscible liquid vehicles such as the lower molecular weight polyethylene glycols are more common due to fewer bioavailability problems.

Available in a wide variety of sizes and shapes, soft shell capsules are both formed, filled, and sealed in the same machine; typically, this is a rotary die process, although a plate process or reciprocating die process also may be employed. Soft shell capsules also may be manufactured in a bubble process that forms seamless spherical capsules. With suitable equipment, powders and other dry solids also may be filled into soft shell capsules.

Liquid-filled capsules of either type involve similar formulation technology and offer similar advantages and limitations. For instance, both may offer advantages over dry-filled capsules and tablets in content uniformity and drug dissolution. Greater homogeneity is possible in liquid systems, and liquids can be metered more accurately. Drug dissolution may benefit because the drug may already be in solution or at least suspended in a hydrophilic vehicle.

Capsules may be coated, or, more commonly, encapsulated granules may be coated to resist releasing the drug in the gastric fluid of the stomach where a delay is important to alleviate potential problems of drug inactivation or gastric mucosal irritation.

In connection with this invention “designed to be orally ingested” is equal to “orally ingestible” and means a formulation which is designed to be taken or consumed orally by a patient and/or if used for a therapeutic use will have to enter the body through the mouth and oesophagus.

In connection with this invention “outwardly solid” means a formulation of which the outer part—the part facing the surrounding—of the formulation is in a solid state prior to and in the immediate beginning of being consumed by a patient.

The phrase “its (tetrodoxin's) derivatives and analogs” according to this invention are defined in part as in U.S. Pat. No. 6,030,974 (incorporated herein in its entirety by reference) as amino perhydroquinazoline compounds having the molecular formula C₁₁H₁₇N₃O₈. Derivatives and analogs of tetrodotoxin according to this invention are further defined in U.S. Pat. No. 5,846,975 (incorporated herein in its entirety by reference) as amino hydrogenated quinazolines and derivatives including the substances described from column 3 line 40 to column 6 line 40. Examples of “derivatives and analogs of tetrodotoxin” according to this invention include, but are not limited to, anhydro-tetrodotoxin, tetrodaminotoxin, methoxytetrodotoxin, ethoxytetrodotoxin, deoxytetrodotoxin and tetrodonic acid, 6 epi-tetrodotoxin, 11-deoxytetrodotoxin as well as the hemilactal type TTX analogs (e.g. 4-epi-TTX, 6-epi-TTX, 11-deoxy-TTX, 4-epi-11-deoxy-TTX, TTX-8-O-hemisuccinate, chiriquitoxin, 11-nor-TTX-6(S)-ol, 11-nor-TTX-6(R)-ol, 11-nor-TTX-6,6-diol, 11-oxo-TTX and TTX-11-carboxylic acid), the lactone type TTX analogs (e.g. 6-epi-TTX (lactone), 11-deoxy-TTX (lactone), 11-nor-TTX-6(S)-ol (lactone), 11-nor-TTX-6(R)-ol (lactone), 11-nor-TTX-6,6-diol (lactone), 5-deoxy-TTX, 5,11-dideoxy-TTX, 4-epi-5,11-didroxy-TTX, 1-hydroxy-5,11-dideoxy-TTX, 5,6,11-trideoxy-TTX and 4-epi-5,6,11-trideoxy-TTX) and the 4,9-anhydro type TTX analogs (e.g. 4,9-anhydro-TTX, 4,9-anhydro-6-epi-TTX, 4,9-anhydro-11-deoxy-TTX, 4,9-anhydro-TTX-8-O-hemisuccinate, 4,9-anhydro-TTX-11-O-hemisuccinate). The typical analogs of TTX possess only ⅛ to 1/40 of the toxicity of TTX in mice, based upon bioassay in mice. It has been observed that the analogs produce joint action, and do not interact adversely. “Joint action” may be either additive or synergistic. Examples of TTX analogs include novel TTX analogs isolated from various organisms, as well as those that are partially or totally chemically synthesized (see e.g., Yotsu, M. et al. Agric. Biol. Chem., 53(3):893-895 (1989)). Analogs of TTX bind to the same site on the alpha subunit of sodium channels as does TTX.

In connection with this invention “neutral form” refers to the non-ionic form but also to (at its isoelectric point) neutrally loaded forms (that means containing an equal amount of positive and negative loads) especially the Zwitter-Ion.

The term “salt” according to this invention is to be understood as meaning any form of the active compound according to the invention in which this compound assumes an ionic form or is charged and—if applicable—is also coupled with a counter-ion (a cation or anion) or is in solution. By this are also to be understood complexes of the active compound with other molecules and ions, in particular complexes which are complexed via ionic interactions. As preferred examples of salts this includes the acetate, mono-trifluoracetate, acetate ester salt, citrate, formate, picrate, hydrobromide, monohydrobromide, monohydrochloride or hydrochloride.

The term “physiologically acceptable salt” in the context of this invention is understood as meaning a “salt” (as defined above) of at least one of the compounds according to the invention which are physiologically tolerated—especially if used in humans and/or mammals.

The term “solvate” according to this invention is to be understood as meaning any form of the active compound according to the invention in which this compound has attached to it via non-covalent binding another molecule (most likely a polar solvent) especially including hydrates and alcoholates, e.g. methanolate.

In connection with this invention any amount defined referred to the compound having a purity of ≧97%. This on the other hand will exclude any impurity contained within the <3% to be mentioned, defined or referred to as active compound in the sense of this invention. For example this would mean that a formulation containing 0.5 mg tetrodotoxin of 99% purity and 0.8% anhydro-tetrodotoxin will be classified and defined according to this invention as containing just tetrodotoxin as active ingredient.

In a highly preferred embodiment of the invention the formulation according to the invention is a solid formulation.

In connection with this invention “solid” means a formulation of which all ingredients—including the outer part—of the formulation are in a solid state in the immediate beginning of being consumed by a patient.

In a preferred embodiment of the invention the formulation according to the invention contains tetrodotoxin and/or any of its analogs in neutral form or in form of a salt.

In a preferred embodiment of the invention the formulation according to the invention contains tetrodotoxin and/or any of its analogs in an amount between 10 μg and 2 mg.

In connection with this invention the “amount” of active ingredient contained refers to each single active compound individually not to the sum of them all being contained.

In a preferred embodiment of the invention the formulation according to the invention contains tetrodotoxin or one of its analogs in neutral form or in form of a salt.

In a preferred embodiment of the invention the formulation according to the invention contains tetrodotoxin or one of its analogs in an amount between 10 μg and 2 mg.

In a preferred embodiment of the invention the formulation according to the invention contains tetrodotoxin in neutral form or in form of a salt.

In a preferred embodiment of the invention the formulation according to the invention contains tetrodotoxin in an amount between 10 μg and 2 mg.

In a preferred embodiment of the invention the formulation according to the invention contains tetrodotoxin isolated from a biological source, preferably from fish, especially puffer fish.

In a preferred embodiment of the invention the formulation according to the invention contains synthesized tetrodotoxin.

In a preferred embodiment of the invention the formulation according to the invention is in form of a tablet, a chewable tablet, a capsule, a drop or a dragee, most preferably in form of a tablet, including coated tablets, or a capsule.

In a preferred embodiment of the invention the formulation according to the invention is an immediate release formulation.

In the context of this invention “immediate release formulation” means any formulation with a release profile from which measured according to a standard measurement (e.g. using the paddle method according to the Pharmacopeia) (e.g. in 0.1% NaCl solution) within 30 minutes more than 50%, more preferably 60%, or even more preferably 70% of the active compound is released.

In a preferred embodiment of the invention the formulation according to the invention is a controlled release formulation.

In the context of this invention “controlled release formulation” which is to be used synonymously with “slow release formulation” and “timed release formulation” means any formulation with a release profile from which measured according to a standard measurement (e.g. using the paddle method according to the Pharmacopeia) (e.g. in 0.1% NaCl solution) within 30 minutes less than 50%, more preferably less than 40%, or even more preferably less than 30% of the active compound is released.

In a preferred embodiment of the invention the formulation according to the invention contains lactose, including hydrates of lactose.

In a preferred embodiment of the invention the formulation according to the invention contains a salt of stearic acid, including magnesium stearate, or sodium stearate.

In a preferred embodiment of the invention the formulation according to the invention contains microcrystalline cellulose.

In a preferred embodiment of the invention the formulation according to the invention contains croscarmelose, including sodium croscarmelose.

In a preferred embodiment of the invention the formulation according to the invention contains colloidal silica(on) dioxide

In a preferred embodiment of the invention the formulation according to the invention contains polyethylene glycol.

The examples in the following section are merely illustrative and the invention cannot be considered in any way as being restricted to these applications. The scope of the invention is limited only by the claims following.

### EXAMPLES

Generally the specific and practical ways of preparing the pharmaceutical formulations exemplified below (as well as that of all other formulations mentioned in this invention) are well known in the art. Accordingly it is i.a. referred to “Remington, the Science and Practice of Pharmacy”, 19^{th} ed., A. R Gennaro ed., c. 1995 by the Philadelphia College of Pharmacy and Science, hereby incorporated in its entirety and for all purposes by reference.

In the formulation Examples described below, certain materials are referred to by trade names. In this regard:

POVIDONE K-30 is manufactured by GAF and is a polyvinylpyrrolidone (PVP) of a mean molecular weight of 30,000.

OPADRY II is distributed by Colorcon and is a mixture of polymers, plasticizers and color pigments.

NATROSOL 250 HHX is a hydroxyethylcellulose product of Hercules, Inc., Wilmington, Del. 250 HHX is a grade that is used in long acting tablet formulations.

CAB-O-SIL is an amorphous fumed silica produced by Cabot Corp. Cabosil is an extremely fine particle size silica (silicon-dioxide/SiO₂) aerogel. It is pure white and free-flowing. Each volume contains about 94% dead air space, with a density of only 2.3 lb/cu ft. On the other hand, water (density 62.4 lb/cu ft) weighs about 27 times more. M5 is a pharmaceutical grade that is a micronized powder.

SURELEASE is a product of Colorcon, West Point, Pa. and is an aqueous ethylcellulose dispersion.

SURETERIC is a product of Colorcon and is an alternative to acrylic polymer systems for enteric coating of solid oral dosage. SURETERIC is a specially blended combination of PVAP (polyvinyl acetate phthalate), plasticizers, and other ingredients in a completely optimized dry powder formulation.

ACRYL-EZE is a product of Colorcon and is an aqueous acrylic enteric coating. Simulated intestinal fluid is described in the U.S. Pharmacoepia and is made by dissolving 6.8 g of monobasic potassium phosphate in 250 ml of water. Then 77 ml of 0.2 N potassium hydroxide is added with 500 ml of water. 10.0 g of pancreatin is added and the solution is adjusted to pH 6.8±0.1 with 0.2 N potassium hydroxide or 0.2 N hydrochloric acid. The volume of the solution is then made to 1 L with water.

Simulated gastric fluid is described in the U.S. Pharmacoepia and is made by dissolving 2.0 g of sodium chloride and 3.2 g of purified pepsin from porcine stomach mucosa and having an activity of 800 to 2500 units per mg in 7.0 ml of hydrochloric acid and sufficient water to make 1 L. The solution has a pH of about 1.2.

### Example 1

### Capsule Formulations

Example of a Formulation (A) for a Capsule

| | | |
|---|---|---|
| Tetrodotoxin (TTX) (powdered material) | 0.03 | mg |
| Colloidal silicon dioxide | 0.5 | mg |
| Magnesium stearate | 1.0 | mg |
| Lactose | 98.47 | mg |
| Total | 100 | mg |

Example of a Formulation (B) for a Capsule

| | | |
|---|---|---|
| Tetrodotoxin (TTX) (powdered material) | 0.15 | mg |
| Colloidal silicon dioxide | 0.5 | mg |
| Magnesium stearate | 1.0 | mg |
| Lactose | 98.35 | mg |
| Total | 100 | mg |

Example of a Formulation (C) for a Capsule

| | | |
|---|---|---|
| Tetrodotoxin (TTX) (powdered material) | 0.3 | mg |
| Colloidal silicon dioxide | 0.5 | mg |
| Magnesium stearate | 1.0 | mg |
| Lactose | 98.2 | mg |
| Total | 100 | mg |

Example of a Formulation (D) for a Capsule

| | | |
|---|---|---|
| Tetrodotoxin (TTX) (powdered material) | 0.9 | mg |
| Colloidal silicon dioxide | 0.5 | mg |
| Magnesium stearate | 1.0 | mg |
| Lactose | 97.6 | mg |
| Total | 100 | mg |

Example of a Formulation (E) for a Capsule

| | | |
|---|---|---|
| Tetrodotoxin (TTX) (powdered material) | 0.25 | mg |
| Colloidal silicon dioxide | 0.5 | mg |
| Magnesium stearate | 1.0 | mg |
| Lactose | 98.25 | mg |
| Total | 100 | mg |

Example of a Formulation (F) for a Capsule

| | | |
|---|---|---|
| Tetrodotoxin (TTX) (powdered material) | 0.5 | mg |
| Colloidal silicon dioxide | 0.5 | mg |
| Magnesium stearate | 1.0 | mg |
| Lactose | 98.0 | mg |
| Total | 100 | mg |

Example of a Formulation (G) for a Capsule

| | | |
|---|---|---|
| Tetrodotoxin (TTX) (powdered material) | 1.0 | mg |
| Colloidal silicon dioxide | 0.5 | mg |
| Magnesium stearate | 1.0 | mg |
| Lactose | 97.5 | mg |
| Total | 100 | mg |

Example of a Formulation (H) for a Capsule

| | | |
|---|---|---|
| Tetrodotoxin (TTX) (powdered material) | 1.5 | mg |
| Colloidal silicon dioxide | 0.5 | mg |
| Magnesium stearate | 1.0 | mg |
| Lactose | 97.0 | mg |
| Total | 100 | mg |

### Example 2

### Tablet Formulations

Example of a Formulation (A) for a Tablet

| | | |
|---|---|---|
| Tetrodotoxin (TTX) (powdered material) | 0.03 | mg |
| Colloidal silicon dioxide | 0.5 | mg |
| Magnesium stearate | 1.0 | mg |
| Sodium croscarmelose | 5.0 | mg |
| Lactose | 93.47 | mg |
| Total | 100 | mg |

Example of a Formulation (B) for a Tablet

| | | |
|---|---|---|
| Tetrodotoxin (TTX) (powdered material) | 0.15 | mg |
| Colloidal silicon dioxide | 0.5 | mg |
| Magnesium stearate | 1.0 | mg |
| Sodium croscarmelose | 5.0 | mg |
| Lactose | 93.35 | mg |
| Total | 100 | mg |

Example of a Formulation (C) for a Tablet

| | | |
|---|---|---|
| Tetrodotoxin (TTX) (powdered material) | 0.3 | mg |
| Colloidal silicon dioxide | 0.5 | mg |
| Magnesium stearate | 1.0 | mg |
| Sodium croscarmelose | 5.0 | mg |
| Lactose | 93.2 | mg |
| Total | 100 | mg |

Example of a Formulation (D) for a Tablet

| | | |
|---|---|---|
| Tetrodotoxin (TTX) (powdered material) | 0.9 | mg |
| Colloidal silicon dioxide | 0.5 | mg |
| Magnesium stearate | 1.0 | mg |
| Sodium croscarmelose | 5.0 | mg |
| Lactose | 92.6 | mg |
| Total | 100 | mg |

Example of a Formulation (E) for a Tablet

| | | |
|---|---|---|
| Tetrodotoxin (TTX) (powdered material) | 0.25 | mg |
| Colloidal silicon dioxide | 0.5 | mg |
| Magnesium stearate | 1.0 | mg |
| Sodium croscarmelose | 5.0 | mg |
| Lactose | 93.25 | mg |
| Total | 100 | mg |

Example of a Formulation (F) for a Tablet

| | | |
|---|---|---|
| Tetrodotoxin (TTX) (powdered material) | 0.5 | mg |
| Colloidal silicon dioxide | 0.5 | mg |
| Magnesium stearate | 1.0 | mg |
| Sodium croscarmelose | 5.0 | mg |
| Lactose | 93.0 | mg |
| Total | 100 | mg |

Example of a Formulation (G) for a Tablet

| | | |
|---|---|---|
| Tetrodotoxin (TTX) (powdered material) | 1.0 | mg |
| Colloidal silicon dioxide | 0.5 | mg |
| Magnesium stearate | 1.0 | mg |
| Sodium croscarmelose | 5.0 | mg |
| Lactose | 92.5 | mg |
| Total | 100 | mg |

Example of a Formulation (H) for a Tablet

| | | |
|---|---|---|
| Tetrodotoxin (TTX) (powdered material) | 1.5 | mg |
| Colloidal silicon dioxide | 0.5 | mg |
| Magnesium stearate | 1.0 | mg |
| Sodium croscarmelose | 5.0 | mg |
| Lactose | 92.0 | mg |
| Total | 100 | mg |

### Example 3

### Additional Tablet Formulations

Example of a Formulation (A) for a Tablet (Humid Granulation)

| | | |
|---|---|---|
| Tetrodotoxin (TTX) (powdered material) | 0.03 | mg |
| Colloidal silicon dioxide | 0.5 | mg |
| Magnesium stearate | 1.0 | mg |
| POVIDONE K-30 | 5.0 | mg |
| Sodium carboxymethylstarch | 5.0 | mg |
| Microcrystalline cellulose | 20 | mg |
| Lactose | 68.47 | mg |
| Total | 100 | mg |

Example of a Formulation (B) for a Tablet (Humid Granulation)

| | | |
|---|---|---|
| Tetrodotoxin (TTX) (powdered material) | 0.15 | mg |
| Colloidal silicon dioxide | 0.5 | mg |
| Magnesium stearate | 1.0 | mg |
| POVIDONE K-30 | 5.0 | mg |
| Sodium carboxymethylstarch | 5.0 | mg |
| Microcrystalline cellulose | 20 | mg |
| Lactose | 68.35 | mg |
| Total | 100 | mg |

Example of a Formulation (C) for a Tablet (Humid Granulation)

| | | |
|---|---|---|
| Tetrodotoxin (TTX) (powdered material) | 0.3 | mg |
| Colloidal silicon dioxide | 0.5 | mg |
| Magnesium stearate | 1.0 | mg |
| POVIDONE K-30 | 5.0 | mg |
| Sodium carboxymethylstarch | 5.0 | mg |
| Microcrystalline cellulose | 20 | mg |
| Lactose | 68.2 | mg |
| Total | 100 | mg |

Example of a Formulation (D) for a Tablet (Humid Granulation)

| | | |
|---|---|---|
| Tetrodotoxin (TTX) (powdered material) | 0.9 | mg |
| Colloidal silicon dioxide | 0.5 | mg |
| Magnesium stearate | 1.0 | mg |
| POVIDONE K-30 | 5.0 | mg |
| Sodium carboxymethylstarch | 5.0 | mg |
| Microcrystalline cellulose | 20 | mg |
| Lactose | 67.6 | mg |
| Total | 100 | mg |

Example of a Formulation (E) for a Tablet (Humid Granulation)

| | | |
|---|---|---|
| Tetrodotoxin (TTX) (powdered material) | 0.25 | mg |
| Colloidal silicon dioxide | 0.5 | mg |
| Magnesium stearate | 1.0 | mg |
| POVIDONE K-30 | 5.0 | mg |
| Sodium carboxymethylstarch | 5.0 | mg |
| Microcrystalline cellulose | 20 | mg |
| Lactose | 68.25 | mg |
| Total | 100 | mg |

Example of a Formulation (F) for a Tablet (Humid Granulation)

| | | |
|---|---|---|
| Tetrodotoxin (TTX) (powdered material) | 0.5 | mg |
| Colloidal silicon dioxide | 0.5 | mg |
| Magnesium stearate | 1.0 | mg |
| POVIDONE K-30 | 5.0 | mg |
| Sodium carboxymethylstarch | 5.0 | mg |
| Microcrystalline cellulose | 20 | mg |
| Lactose | 68.0 | mg |
| Total | 100 | mg |

Example of a Formulation (G) for a Tablet (Humid Granulation)

| | | |
|---|---|---|
| Tetrodotoxin (TTX) (powdered material) | 1.0 | mg |
| Colloidal silicon dioxide | 0.5 | mg |
| Magnesium stearate | 1.0 | mg |
| POVIDONE K-30 | 5.0 | mg |
| Sodium carboxymethylstarch | 5.0 | mg |
| Microcrystalline cellulose | 20 | mg |
| Lactose | 67.5 | mg |
| Total | 100 | mg |

Example of a Formulation (H) for a Tablet (Humid Granulation)

| | | |
|---|---|---|
| Tetrodotoxin (TTX) (powdered material) | 1.5 | mg |
| Colloidal silicon dioxide | 0.5 | mg |
| Magnesium stearate | 1.0 | mg |
| POVIDONE K-30 | 5.0 | mg |
| Sodium carboxymethylstarch | 5.0 | mg |
| Microcrystalline cellulose | 20 | mg |
| Lactose | 67.0 | mg |
| Total | 100 | mg |

### Example 4

### Additional Tablet Formulations

Example of a Formulation (A) for a Tablet

| | | |
|---|---|---|
| Tetrodotoxin (TTX) (powdered material) | 0.03 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (AVICEL PH-102) | 346 | mg |
| Lactose monohydrate (FARMATOSE 200M) | 365.97 | mg |
| Total | 800 | mg |

Example of a Formulation (B) for a Tablet

| | | |
|---|---|---|
| Tetrodotoxin (TTX) (powdered material) | 0.06 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (AVICEL PH-102) | 346 | mg |
| Lactose monohydrate (FARMATOSE 200M) | 365.94 | mg |
| Total | 800 | mg |

Example of a Formulation (C) for a Tablet

| | | |
|---|---|---|
| Tetrodotoxin (TTX) (powdered material) | 0.12 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (Avicel PH-102) | 346 | mg |
| Lactose monohydrate (FARMATOSE 200M) | 365.88 | mg |
| Total | 800 | mg |

Example of a Formulation (D) for a Tablet

| | | |
|---|---|---|
| Tetrodotoxin (TTX) (powdered material) | 0.18 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (Avicel PH-102) | 346 | mg |
| Lactose monohydrate (FARMATOSE 200M) | 365.82 | mg |
| Total | 800 | mg |

Example of a Formulation (E) for a Tablet

| | | |
|---|---|---|
| Tetrodotoxin (TTX) (powdered material) | 0.3 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (Avicel PH-102) | 346 | mg |
| Lactose monohydrate (FARMATOSE 200M) | 365.7 | mg |
| Total | 800 | mg |

Example of a Formulation (F) for a Tablet

| | | |
|---|---|---|
| Tetrodotoxin (TTX) (powdered material) | 0.9 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (Avicel PH-102) | 346 | mg |
| Lactose monohydrate (FARMATOSE 200M) | 365.1 | mg |
| Total | 800 | mg |

Example of a Formulation (G) for a Tablet

| | | |
|---|---|---|
| Tetrodotoxin (TTX) (powdered material) | 0.25 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (Avicel PH-102) | 346 | mg |
| Lactose monohydrate (FARMATOSE 200M) | 365.75 | mg |
| Total | 800 | mg |

Example of a Formulation (H) for a Tablet

| | | |
|---|---|---|
| Tetrodotoxin (TTX) (powdered material) | 0.5 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (Avicel PH-102) | 346 | mg |
| Lactose monohydrate (FARMATOSE 200M) | 365.5 | mg |
| Total | 800 | mg |

Example of a Formulation (I) for a Tablet

| | | |
|---|---|---|
| Tetrodotoxin (TTX) (powdered material) | 1.0 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (Avicel PH-102) | 346 | mg |
| Lactose monohydrate (FARMATOSE 200M) | 365.0 | mg |
| Total | 800 | mg |

Example of a Formulation (J) for a Tablet

| | | |
|---|---|---|
| Tetrodotoxin (TTX) (powdered material) | 1.5 | mg |
| Sodium croscarmelose (AC-DI-SOL) | 40 | mg |
| Colloidal silica dioxide (AEROSYL 200) | 8 | mg |
| Magnesium stearate, NF | 16 | mg |
| POVIDONE K-30 | 40 | mg |
| Microcrystalline cellulose (Avicel PH-102) | 346 | mg |
| Lactose monohydrate (FARMATOSE 200M) | 364.5 | mg |
| Total | 800 | mg |

### Example 5

### Additional Tablet Formulations

Example of an Alternative Formulation (A) for a Tablet

| | | |
|---|---|---|
| Tetrodotoxin (TTX) (powdered material) | 0.03 | mg |
| Sodium croscarmelose (AC-DI-SOL) | 35 | mg |
| Colloidal silica dioxide (AEROSYL 200) | 3 | mg |
| Sodium stearate | 12 | mg |
| Polyethylene glycol 8000 | 30 | mg |
| Microcrystalline cellulose (Avicel PH-102) | 75 | mg |
| Lactose monohydrate (FARMATOSE 200M) | 420.97 | mg |
| OPADRY II ® | 24 | mg |
| Total | 600 | mg |

Example of an Alternative Formulation (B) for a Tablet

| | | |
|---|---|---|
| Tetrodotoxin (TTX) (powdered material) | 0.15 | mg |
| Sodium croscarmelose (AC-DI-SOL) | 35 | mg |
| Colloidal silica dioxide (AEROSYL 200) | 3 | mg |
| Sodium stearate | 12 | mg |
| Polyethylene glycol 8000 | 30 | mg |
| Microcrystalline cellulose (Avicel PH-102) | 75 | mg |
| Lactose monohydrate (FARMATOSE 200M) | 420.85 | mg |
| OPADRY II ® | 24 | mg |
| Total | 600 | mg |

Example of an Alternative Formulation (C) for a Tablet

| | | |
|---|---|---|
| Tetrodotoxin (TTX) (powdered material) | 0.3 | mg |
| Sodium croscarmelose (AC-DI-SOL) | 35 | mg |
| Colloidal silica dioxide (AEROSYL 200) | 3 | mg |
| Sodium stearate | 12 | mg |
| Polyethylene glycol 8000 | 30 | mg |
| Microcrystalline cellulose (Avicel PH-102) | 75 | mg |
| Lactose monohydrate (FARMATOSE 200M) | 420.7 | mg |
| OPADRY II ® | 24 | mg |
| Total | 600 | mg |

Example of an Alternative Formulation (D) for a Tablet

| | | |
|---|---|---|
| Tetrodotoxin (TTX) (powdered material) | 0.9 | mg |
| Sodium croscarmelose (AC-DI-SOL) | 35 | mg |
| Colloidal silica dioxide (AEROSYL 200) | 3 | mg |
| Sodium stearate | 12 | mg |
| Polyethylene glycol 8000 | 30 | mg |
| Microcrystalline cellulose (Avicel PH-102) | 75 | mg |
| Lactose monohydrate (FARMATOSE 200M) | 420.1 | mg |
| OPADRY II ® | 24 | mg |
| Total | 600 | mg |

Example of an Alternative Formulation (E) for a Tablet

| | | |
|---|---|---|
| Tetrodotoxin (TTX) (powdered material) | 0.25 | mg |
| Sodium croscarmelose (AC-DI-SOL) | 35 | mg |
| Colloidal silica dioxide (AEROSYL 200) | 3 | mg |
| Sodium stearate | 12 | mg |
| Polyethylene glycol 8000 | 30 | mg |
| Microcrystalline cellulose (Avicel PH-102) | 75 | mg |
| Lactose monohydrate (FARMATOSE 200M) | 420.75 | mg |
| OPADRY II ® | 24 | mg |
| Total | 600 | mg |

Example of an Alternative Formulation (F) for a Tablet

| | | |
|---|---|---|
| Tetrodotoxin (TTX) (powdered material) | 0.5 | mg |
| Sodium croscarmelose (AC-DI-SOL) | 35 | mg |
| Colloidal silica dioxide (AEROSYL 200) | 3 | mg |
| Sodium stearate | 12 | mg |
| Polyethylene glycol 8000 | 30 | mg |
| Microcrystalline cellulose (Avicel PH-102) | 75 | mg |
| Lactose monohydrate (FARMATOSE 200M) | 420.5 | mg |
| OPADRY II ® | 24 | mg |
| Total | 600 | mg |

Example of an Alternative Formulation (G) for a Tablet

| | | |
|---|---|---|
| Tetrodotoxin (TTX) (powdered material) | 1.0 | mg |
| Sodium croscarmelose (AC-DI-SOL) | 35 | mg |
| Colloidal silica dioxide (AEROSYL 200) | 3 | mg |
| Sodium stearate | 12 | mg |
| Polyethylene glycol 8000 | 30 | mg |
| Microcrystalline cellulose (Avicel PH-102) | 75 | mg |
| Lactose monohydrate (FARMATOSE 200M) | 420.0 | mg |
| OPADRY II ® | 24 | mg |
| Total | 600 | mg |

Example of an Alternative Formulation (H) for a Tablet

| | | |
|---|---|---|
| Tetrodotoxin (TTX) (powdered material) | 1.5 | mg |
| Sodium croscarmelose (AC-DI-SOL) | 35 | mg |
| Colloidal silica dioxide (AEROSYL 200) | 3 | mg |
| Sodium stearate | 12 | mg |
| Polyethylene glycol 8000 | 30 | mg |
| Microcrystalline cellulose (Avicel PH-102) | 75 | mg |
| Lactose monohydrate (FARMATOSE 200M) | 419.5 | mg |
| OPADRY II ® | 24 | mg |
| Total | 600 | mg |

### Example 6

### Additional Capsule Formulations

Example of an Alternative Formulation (A) of a Capsule

| | | |
|---|---|---|
| Tetrodotoxin | 0.03 | mg |
| Colloidal silica dioxide | 0.8 | mg |
| Magnesium stearate | 2.4 | mg |
| Lactose | 476.77 | mg |
| Total | 480 | mg |

Example of an Alternative Formulation (B) of a Capsule

| | | |
|---|---|---|
| Tetrodotoxin | 0.15 | mg |
| Colloidal silica dioxide | 0.8 | mg |
| Magnesium stearate | 2.4 | mg |
| Lactose | 476.65 | mg |
| Total | 480 | mg |

Example of an Alternative Formulation (C) of a Capsule

| | | |
|---|---|---|
| Tetrodotoxin | 0.3 | mg |
| Colloidal silica dioxide | 0.8 | mg |
| Magnesium stearate | 2.4 | mg |
| Lactose | 476.5 | mg |
| Total | 480 | mg |

Example of an Alternative Formulation (D) of a Capsule

| | | |
|---|---|---|
| Tetrodotoxin | 0.9 | mg |
| Colloidal silica dioxide | 0.8 | mg |
| Magnesium stearate | 2.4 | mg |
| Lactose | 475.9 | mg |
| Total | 480 | mg |

Example of an Alternative Formulation (E) of a Capsule

| | | |
|---|---|---|
| Tetrodotoxin | 0.25 | mg |
| Colloidal silica dioxide | 0.8 | mg |
| Magnesium stearate | 2.4 | mg |
| Lactose | 476.55 | mg |
| Total | 480 | mg |

Example of an Alternative Formulation (F) of a Capsule

| | | |
|---|---|---|
| Tetrodotoxin | 0.5 | mg |
| Colloidal silica dioxide | 0.8 | mg |
| Magnesium stearate | 2.4 | mg |
| Lactose | 476.3 | mg |
| Total | 480 | mg |

Example of an Alternative Formulation (G) of a Capsule

| | | |
|---|---|---|
| Tetrodotoxin | 1.0 | mg |
| Colloidal silica dioxide | 0.8 | mg |
| Magnesium stearate | 2.4 | mg |
| Lactose | 475.8 | mg |
| Total | 480 | mg |

Example of an Alternative Formulation (H) of a Capsule

| | | |
|---|---|---|
| Tetrodotoxin | 1.5 | mg |
| Colloidal silica dioxide | 0.8 | mg |
| Magnesium stearate | 2.4 | mg |
| Lactose | 475.3 | mg |
| Total | 480 | mg |

### Example 7

### Outwardly Solid Formulations

Encapsulated Outwardly Solid Formulation (A)

| | | |
|---|---|---|
| Tetrodotoxin | 60 | mg |
| 0.5% dilute acetic acid | 1 | ml |
| Acetic Acid-acetate buffer solution | 50 | ml (5% of the total volume of |
| (pH = 3.5) | | the prepared pharmaceutical |
| | | solution) |
| Water, sterile, add to | 1000 | ml |

0.5 ml of this prepared solution were encapsulated in suitable consumable capsules and stored.

Encapsulated Outwardly Solid Formulation (B):

| | | |
|---|---|---|
| Tetrodotoxin | 300 | mg |
| 0.5% dilute acetic acid | 1 | ml |
| Acetic Acid-acetate buffer solution | 50 | ml (5% of the total volume of |
| (pH = 3.5) | | the prepared pharmaceutical |
| | | solution) |
| Water, sterile, add to | 1000 | ml |

0.5 ml of this prepared solution were encapsulated in suitable consumable capsules and stored.

Encapsulated Outwardly Solid Formulation (C)

| | | |
|---|---|---|
| Tetrodotoxin | 600 | mg |
| 0.5% dilute acetic acid | 1 | ml |
| Acetic Acid-acetate buffer solution | 50 | ml (5% of the total volume of |
| (pH = 3.5) | | the prepared pharmaceutical |
| | | solution) |
| Water, sterile, add to | 1000 | ml |

0.5 ml of this prepared solution were encapsulated in suitable consumable capsules and stored.

Encapsulated Outwardly Solid Formulation (D)

| | | |
|---|---|---|
| Tetrodotoxin | 1800 | mg |
| 0.5% dilute acetic acid | 1 | ml |
| Acetic Acid-acetate buffer solution | 50 | ml (5% of the total volume of |
| (pH = 3.5) | | the prepared pharmaceutical |
| | | solution) |
| Water, sterile, add to | 1000 | ml |

0.5 ml of this prepared solution were encapsulated in suitable consumable capsules and stored.

Encapsulated Outwardly Solid Formulation (E)

| | | |
|---|---|---|
| Tetrodotoxin | 500 | mg |
| 0.5% dilute acetic acid | 1 | ml |
| Acetic Acid-acetate buffer solution | 50 | ml (5% of the total volume of |
| (pH = 3.5) | | the prepared pharmaceutical |
| | | solution) |
| Water, sterile, add to | 1000 | ml |

0.5 ml of this prepared solution were encapsulated in suitable consumable capsules and stored.

Encapsulated Outwardly Solid Formulation (F)

| | | |
|---|---|---|
| Tetrodotoxin | 1000 | mg |
| 0.5% dilute acetic acid | 1 | ml |
| Acetic Acid-acetate buffer solution | 50 | ml (5% of the total volume of |
| (pH = 3.5) | | the prepared pharmaceutical |
| | | solution) |
| Water, sterile, add to | 1000 | ml |

0.5 ml of this prepared solution were encapsulated in suitable consumable capsules and stored.

Encapsulated Outwardly Solid Formulation (G)

| | | |
|---|---|---|
| Tetrodotoxin | 2000 | mg |
| 0.5% dilute acetic acid | 1 | ml |
| Acetic Acid-acetate buffer solution | 50 | ml (5% of the total volume of |
| (pH = 3.5) | | the prepared pharmaceutical |
| | | solution) |
| Water, sterile, add to | 1000 | ml |

0.5 ml of this prepared solution were encapsulated in suitable consumable capsules and stored.

Encapsulated Outwardly Solid Formulation (H)

| | | |
|---|---|---|
| Tetrodotoxin | 3000 | mg |
| 0.5% dilute acetic acid | 1 | ml |
| Acetic Acid-acetate buffer solution | 50 | ml (5% of the total volume of |
| (pH = 3.5) | | the prepared pharmaceutical |
| | | solution) |
| Water, sterile, add to | 1000 | ml |

0.5 ml of this prepared solution were encapsulated in suitable consumable capsules and stored.

### Example 8

Example of a Further Alternative Formulation of a Tablet Ready to be Processed into an Enteric-Coated Formulation

| | | |
|---|---|---|
| Tetrodotoxin | 0.5 | mg |
| Dibasic Calcium Phosphate USP | 46.8 | mg |
| Avicel PH 101 | 50.0 | mg |
| NATROSOL 250 HHX | 1.0 | mg |
| CAB-O-SIL M5 | 0.5 | mg |
| Magnesium Stearate NF | 1.0 | mg |
| Yellow Lake F D & C No 6 | 0.2 | mg |
| Purified Water USP (evaporates during the process) | | |
| Total | 100 | mg |

### Example 9

Example of an Enteric-Coated Version of Example 8

| | |
|---|---|
| Tablet according to Example 8 | 100 mg |
| Acryl-Eze yellow coating suspension House Std | 40.0 mg |

### Example 10

Example of Another Form of Tablet Ready to be Processed into a Coated Controlled-Release Formulation

| | | |
|---|---|---|
| Tetrodotoxin | 0.5 | mg |
| Dibasic Calcium Phosphate USP | 40.0 | mg |
| Avicel PH 101 | 46.8 | mg |
| NATROSOL 250 HHX | 10.0 | mg |
| CAB-O-SIL M5 | 0.5 | mg |
| Magnesium Stearate NF | 2.0 | mg |
| Blue F D & C No1 | 0.2 | mg |
| Purified Water USP (evaporates during the process) | | |
| Total | 100 | mg |

### Example 11

Example of a Coated Controlled-Release Version of Example 10

| | |
|---|---|
| Tablet according to Example 10 | 100 mg |
| SURETERIC Blue suspension House Std | 20.0 mg |
| 90/10 SURELEASE/OPADRY clear suspension | 30.0 mg |

### Example 12

Example of a Further Alternative Formulation of a Tablet Ready to be Processed into a Coated Formulation

| | | |
|---|---|---|
| Tetrodotoxin | 0.5 | mg |
| Dibasic Calcium Phosphate USP | 46.0 | mg |
| Avicel PH 101 | 50.0 | mg |
| AC-DI-SOL | 2.0 | mg |
| CAB-O-SIL M5 | 0.5 | mg |
| Magnesium Stearate NF | 1.0 | mg |
| Purified Water USP (evaporates during the process) | | |
| Total | 100 | mg |

### Example 13

Example of a Coated Version of Example 12

| | |
|---|---|
| Tablet according to Example 12 | 100 mg |
| OPADRY II coating suspension House Std | 20.0 mg |

### Example 14

Example of a Further Alternative Formulation of a Tablet Ready to be Processed into an Enteric-Coated Formulation

| | | |
|---|---|---|
| Tetrodotoxin | 1.0 | mg |
| Dibasic Calcium Phosphate USP | 46.3 | mg |
| Avicel PH 101 | 50.0 | mg |
| NATROSOL 250 HHX | 1.0 | mg |
| CAB-O-SIL M5 | 0.5 | mg |
| Magnesium Stearate NF | 1.0 | mg |
| Yellow Lake F D & C No 6 | 0.2 | mg |
| Purified Water USP (evaporates during the | | |
| process) | | |
| Total | 100 | mg |

### Example 15

Example of an Enteric-Coated Version of Example 14

| | | |
|---|---|---|
| Tablet according to Example 14 | 100 | mg |
| Acryl-Eze yellow coating suspension House Std | 40.0 | mg |

### Example 16

Example of Another Form of Tablet Ready to be Processed into a Coated Controlled-Release Formulation

| | | |
|---|---|---|
| Tetrodotoxin | 1.0 | mg |
| Dibasic Calcium Phosphate USP | 40.0 | mg |
| Avicel PH 101 | 46.3 | mg |
| NATROSOL 250 HHX | 10.0 | mg |
| CAB-O-SIL M5 | 0.5 | mg |
| Magnesium Stearate NF | 2.0 | mg |
| Blue F D & C No1 | 0.2 | mg |
| Purified Water USP (evaporates during the process) | | |
| Total | 100 | mg |

### Example 17

Example of a Coated Controlled-Release Version of Example 16

| | | |
|---|---|---|
| Tablet according to Example 16 | 100 | mg |
| SURETERIC Blue suspension House Std | 20.0 | mg |
| 90/10 SURELEASE/OPADRY clear suspension | 30.0 | mg |

### Example 18

Example of a Further Alternative Formulation of a Tablet Ready to be Processed into a Coated Formulation

| | | |
|---|---|---|
| Tetrodotoxin | 1.0 | mg |
| Dibasic Calcium Phosphate USP | 45.5 | mg |
| Avicel PH 101 | 50.0 | mg |
| AC-DI-SOL | 2.0 | mg |
| CAB-O-SIL M5 | 0.5 | mg |
| Magnesium Stearate NF | 1.0 | mg |
| Purified Water USP (evaporates during the process) | | |
| Total | 100 | mg |

### Example 19

Example of a Coated Version of Example 18

| | |
|---|---|
| Tablet according to Example 18 | 100 mg |
| OPADRY II coating suspension House Std | 20.0 mg |

### Example 20

Stability of Tetrodotoxin in Gastric Fluid

The stability of tetrodotoxin in gastric fluid was investigated by incubation of tetrodotoxin in freshly prepared Simulated Gastric Fluid at 37° C.±0.5° C. for various times. HPLC analysis showed that the level of tetrodotoxin—opposed to common believe in the art—remains relatively unchanged after 26 hours of incubation, indicating that tetrodotoxin is stable in gastric fluid.

| Incubation Time (hour) | Relative Peak Area (%) |
|---|---|
| 0 | 100.0 |
| 0.5 | 100.0 |
| 1.5 | 100.9 |
| 3 | 100.9 |
| 6 | 99.9 |
| 19 | 99.9 |
| 23 | 98.4 |

### Example 21

Stability of Tetrodotoxin in Intestinal Fluid

The stability of tetrodotoxin in intestinal fluid was investigated by incubation of tetrodotoxin in freshly prepared Simulated Intestinal Fluid at 37° C.±0.5° C. for various times. HPLC analysis showed that up to 5.7% tetrodotoxin was degraded after 8 hours of incubation, indicating that tetrodotoxin is relatively stable in intestinal fluid.

| Incubation Time (hour) | Relative Peak Area (%) |
|---|---|
| 0 | 100.0 |
| 1 | 98.4 |
| 2 | 97.0 |
| 4 | 96.7 |
| 7 | 96.4 |
| 8 | 94.3 |

1. A pharmaceutical composition comprising tetrodotoxin and/or at least one derivative and/or analog thereof in an outwardly solid form suitable for oral ingestion.

2. The pharmaceutical composition of claim 1, in which the tetrodotoxin and/or analog and/or derivative thereof is present as a racemate or as a mixture of stereoisomers.

3. The pharmaceutical composition of claim 1, in which the tetrodotoxin and/or analog and/or derivative thereof is present as a pure stereoisomer.

4. The pharmaceutical composition of claim 1, in which the tetrodotoxin and/or analog and/or derivative thereof is present in neutral form.

5. The pharmaceutical composition of claim 1, in which the tetrodotoxin and/or analog and/or derivative thereof is present in the form of a solvate.

6. The pharmaceutical composition of claim 1 that is a solid formulation.

7. The pharmaceutical composition of claim 6 in which the tetrodotoxin and/or analog or derivative thereof is in neutral form.

8. The pharmaceutical composition of claim 6 in which the tetrodotoxin and/or analog or derivative thereof is in the form of a salt.

9. The pharmaceutical composition of any one of claims 1-8 in which the tetrodotoxin and/or the analog or derivative thereof is present in a total amount between 10 μg and 2 mg.

10. The pharmaceutical composition of claim 1 that comprises tetrodotoxin.

11. The pharmaceutical composition of claim 1 that is in the form of a tablet, a chewable tablet, a capsule, a drop or a dragee.

12. The pharmaceutical composition of any one of claims 1-8 that is formulated as an immediate release formulation.

13. The pharmaceutical composition of claim 12, in which the tetrodotoxin and/or the analog or derivative thereof is present in a total amount between 10 μg and 2 mg.

14. The pharmaceutical composition of any one of claims 1-8 that is formulated as a controlled release formulation.

15. The pharmaceutical composition of claim 14, in which the tetrodotoxin and/or the analog or derivative thereof is present in a total amount between 10 μg and 2 mg.

16. The pharmaceutical composition according to claim 1, further comprising lactose and/or hydrates of lactose.

17. The pharmaceutical composition according to claim 1, further comprising a salt of an organic acid.

18. The pharmaceutical composition of claim 17, in which the salt is a salt of stearic acid.

19. The pharmaceutical composition according to claim 1, further comprising microcrystalline cellulose.

20. The pharmaceutical composition according to claim 1, further comprising croscarmelose.

21. The pharmaceutical composition according to claim 1, further comprising colloidal silicon dioxide.

22. The pharmaceutical composition according to claim 1, further comprising polyethylene glycol.

23. The pharmaceutical composition according to claim 1, further comprising a dibasic phosphate salt.
